(19) United States
(12) Patent Application Publication
(10) Pub. No.: US 2018/0299400 A1
(43) Pub. Date: Oct. 18, 2018

(54) USE OF FLUOROPOLYMERS AS A HYDROPHOBIC LAYER TO SUPPORT LIPID BILAYER FORMATION FOR NANOPORE BASED DNA SEQUENCING

(71) Applicant: Genia Technologies, Inc., Santa Clara, CA (US)

(72) Inventors: John Foster, Mountain View, CA (US); Steven Henck, Mountain View, CA (US)

(21) Appl. No.: 15/768,215

(22) PCT Filed: Oct. 21, 2016
(86) PCT No.: PCT/US16/58230

§ 371 Date: Apr. 13, 2018

Related U.S. Application Data
(60) Provisional application No. 62/244,680, filed on Oct. 21, 2015.

(51) Int. Cl. G01N 27/327; C12Q 1/6869; G01N 33/487; G01N 27/40

(52) U.S. Cl. CPC G01N 27/3278; C12Q 1/6869; G01N 27/40; G01N 27/3276; G01N 33/48721

(57) ABSTRACT

A method of sequencing a DNA sample is disclosed. A nanopore-based sequencing device is provided. The nanopore-based sequencing device includes a conductive layer. The device further includes a working electrode disposed above the conductive layer. The device further includes a side wall disposed above the working electrode, wherein the side wall and the working electrode form a well in which an electrolyte may be contained, and wherein at least an upper portion of the side wall comprises a hydrophobic portion formed by a fluoropolymer material. The DNA sample is sequenced using the nanopore-based sequencing device.

### CROSS REFERENCE TO OTHER APPLICATIONS

This application claims priority to U.S. Provisional Patent Application No. 62/244,680 entitled USE OF FLUOROPOLYMERS AS A HYDROPHOBIC FILM TO SUPPORT LIPID BILAYER FORMATION FOR NANOPORE BASED DNA SEQUENCING filed Oct. 21, 2015 which is incorporated herein by reference for all purposes.

### BACKGROUND OF THE INVENTION

Advances in micro-miniaturization within the semiconductor industry in recent years have enabled biotechnologists to begin packing traditionally bulky sensing tools into smaller and smaller form factors, onto so-called biochips. It would be desirable to develop techniques for biochips that make them more robust, efficient, and cost-effective.

### BRIEF DESCRIPTION OF THE DRAWINGS

Various embodiments of the invention are disclosed in the following detailed description and the accompanying drawings.

FIG. 1 illustrates an embodiment of a cell 100 in a nanopore-based sequencing chip.

FIG. 2 illustrates an embodiment of a cell 200 performing nucleotide sequencing with the Nano-SBS technique.

FIG. 3 illustrates an embodiment of a cell about to perform nucleotide sequencing with pre-loaded tags.

FIG. 4 illustrates an embodiment of a process 400 for nucleic acid sequencing with pre-loaded tags.

FIG. 5 illustrates a cross-sectional view of an embodiment of an electrochemical cell 500 in a nanopore-based sequencing chip that includes a fluoropolymer hydrophobic layer to facilitate the formation of a lipid bilayer.

FIG. 6 illustrates a top view of a plurality of circular openings 602 of a plurality of wells in a nanopore-based sequencing chip.

FIGS. 7A-7H illustrate the various steps of an embodiment of a process 700 for constructing a non-faradaic electrochemical cell of a nanopore-based sequencing chip that includes a fluoropolymer hydrophobic layer to facilitate the formation of a lipid bilayer and a TiN working electrode with increased electrochemical capacitance.

FIG. 8 illustrates a cross-section view of a spongy and porous TiN layer 802 deposited above a metal layer 804.

FIG. 9 illustrates a cross-sectional photograph of an electrochemical cell 900 in a nanopore-based sequencing chip that includes a fluoropolymer hydrophobic layer to facilitate the formation of a lipid bilayer.

FIG. 10 illustrates a cross-sectional view of another embodiment of an electrochemical cell 1000 in a nanopore-based sequencing chip that includes a fluoropolymer hydrophobic layer to facilitate the formation of a lipid bilayer.

FIG. 11 illustrates a cross-sectional view of another embodiment of an electrochemical cell 1100 in a nanopore-based sequencing chip that includes a fluoropolymer hydrophobic layer to facilitate the formation of a lipid bilayer.

FIG. 12 illustrates a cross-sectional view of another embodiment of an electrochemical cell 1200 in a nanopore-based sequencing chip that includes a fluoropolymer hydrophobic layer to facilitate the formation of a lipid bilayer.

FIG. 13 illustrates a cross-sectional view of another embodiment of an electrochemical cell 1300 in a nanopore-based sequencing chip that includes a fluoropolymer hydrophobic layer to facilitate the formation of a lipid bilayer.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Nanopore membrane devices having pore sizes on the order of one nanometer in internal diameter have shown promise in rapid nucleotide sequencing. When a voltage potential is applied across a nanopore immersed in a conducting fluid, a small ion current attributed to the conduction of ions through the nanopore can be observed. The size of the current is sensitive to the pore size.

A nanopore-based sequencing chip may be used for nucleic acid (e.g., DNA) sequencing. A nanopore-based sequencing chip incorporates a large number of sensor cells configured as an array. For example, an array of one million cells may include 1000 rows by 1000 columns of cells.

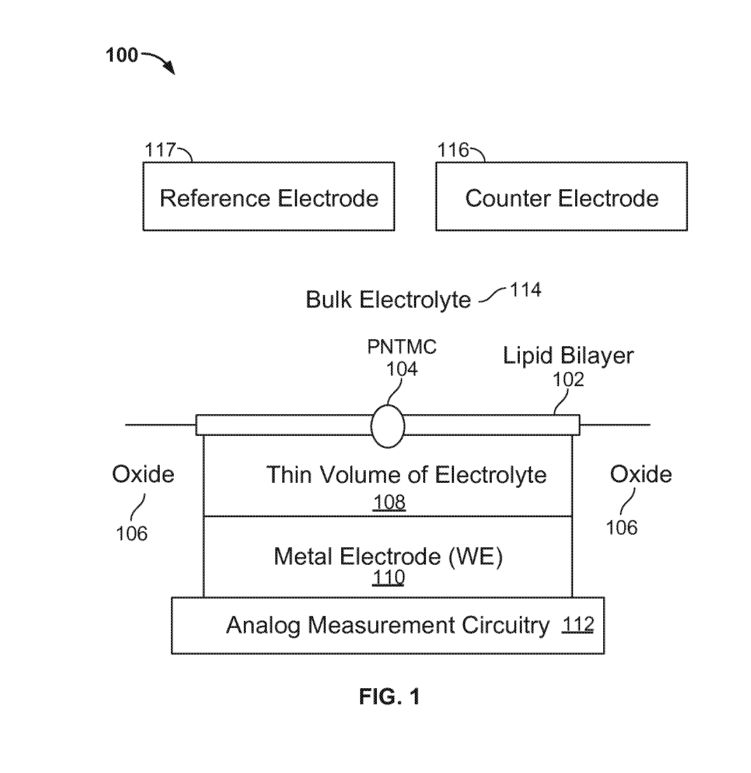

FIG. 1 illustrates an embodiment of a cell 100 in a nanopore-based sequencing chip. A membrane 102 is formed over the surface of the cell. In some embodiments, membrane 102 is a lipid bilayer. The bulk electrolyte 114 containing soluble protein nanopore transmembrane molecular complexes (PNTMC) and the analyte of interest is placed directly onto the surface of the cell. In one embodiment, a single PNTMC 104 is inserted into membrane 102 by electroporation. The individual membranes in the array are neither chemically nor electrically connected to each other. Thus, each cell in the array is an independent sequencing machine, producing data unique to the single polymer molecule associated with the PNTMC. PNTMC 104 operates on the analytes and modulates the ionic current through the otherwise impermeable bilayer.

With continued reference to FIG. 1, analog measurement circuitry 112 is connected to a working electrode 110 covered by a volume of electrolyte 108. The volume of electrolyte 108 is isolated from the bulk electrolyte 114 by the ion-impermeable membrane 102. PNTMC 104 crosses membrane 102 and provides the only path for ionic current to flow from the bulk liquid to working electrode 110. The cell also includes a counter electrode (CE) 116, which is in electrical contact with the bulk electrolyte 114. The cell may also include a reference electrode 117. In one embodiment, the working electrode 110 is a metal electrode. In another embodiment, the electrode comprises a conductive material.

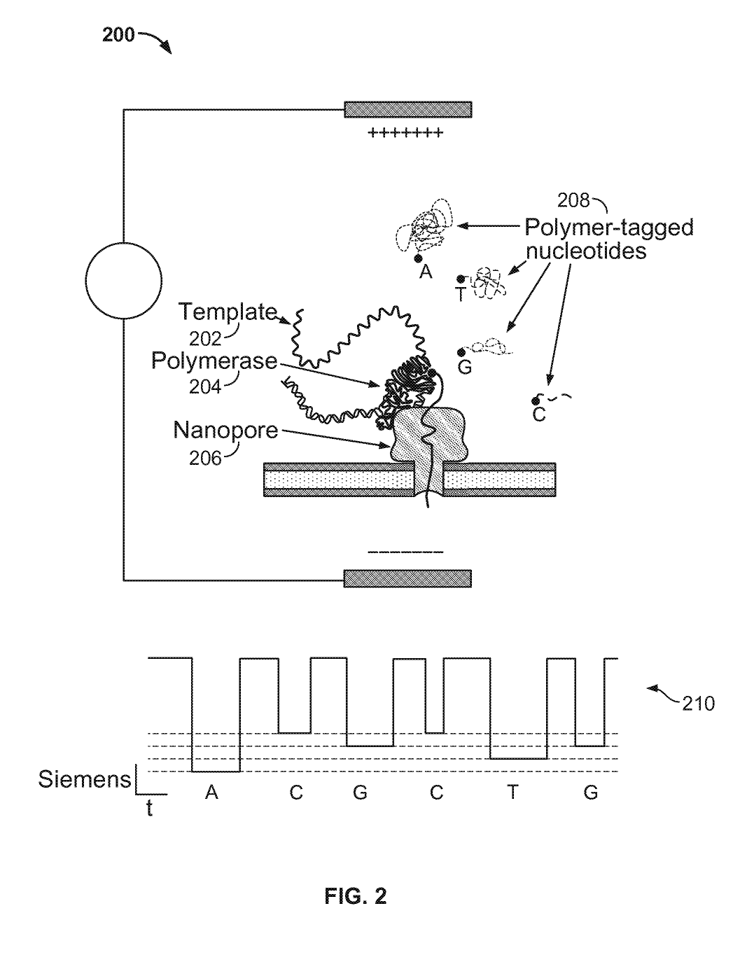

In some embodiments, a nanopore array enables parallel sequencing using the single molecule nanopore-based sequencing by synthesis (Nano-SBS) technique. FIG. 2 illustrates an embodiment of a cell 200 performing nucleotide sequencing with the Nano-SBS technique. In the Nano-SBS technique, a template 202 to be sequenced and a primer are introduced to cell 200. To this template-primer complex, four differently tagged nucleotides 208 are added to the bulk aqueous phase. As the correctly tagged nucleotide is complexed with the polymerase 204, the tail of the tag is positioned in the barrel of nanopore 206. The tag held in the barrel of nanopore 206 generates a unique ionic blockade signal 210, thereby electronically identifying the added base due to the tags' distinct chemical structures.

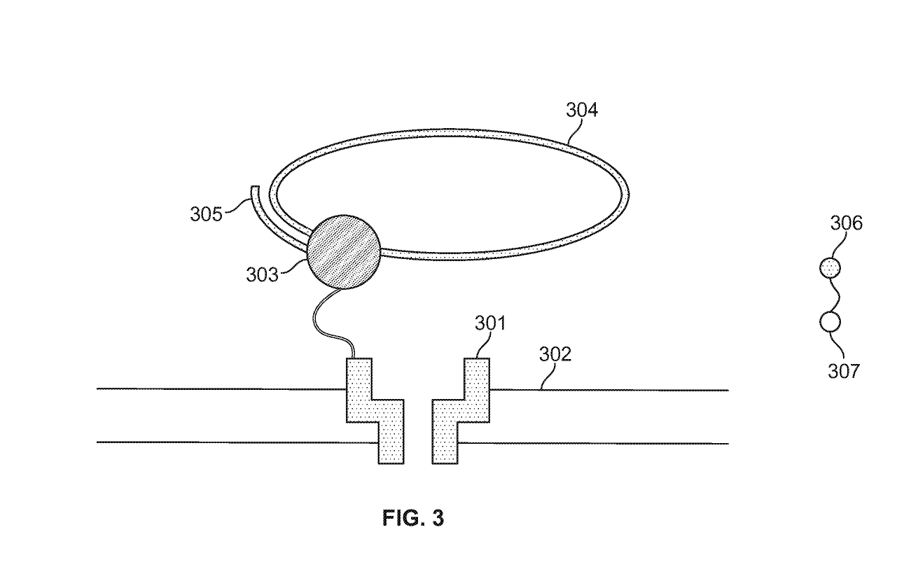

FIG. 3 illustrates an embodiment of a cell about to perform nucleotide sequencing with pre-loaded tags. A nanopore 301 is formed or is inserted in a membrane 302. An enzyme 303 (e.g., a polymerase, such as a DNA polymerase) is associated with the nanopore. In some cases, polymerase 303 is covalently attached to nanopore 301. Polymerase 303 is associated with a nucleic acid molecule 304 to be sequenced. In some embodiments, the nucleic acid molecule 304 is circular. In some cases, nucleic acid molecule 304 is linear. In some embodiments, a nucleic acid primer 305 is hybridized to a portion of nucleic acid molecule 304. Polymerase 303 catalyzes the incorporation of nucleotides 306 onto primer 305 using single stranded nucleic acid molecule 304 as a template. Nucleotides 306 comprise tag species (“tags”) 307.

FIG. 4 illustrates an embodiment of a process 400 for nucleic acid sequencing with pre-loaded tags. Stage A illustrates the components as described in FIG. 3. Stage C shows the tag loaded into the nanopore. A “loaded” tag may be one that is positioned in and/or remains in or near the nanopore for an appreciable amount of time, e.g., 0.1 millisecond (ms) to 10,000 ms. In some cases, a tag that is pre-loaded is loaded in the nanopore prior to being released from the nucleotide. In some instances, a tag is pre-loaded if the probability of the tag passing through (and/or being detected by) the nanopore after being released upon a nucleotide incorporation event is suitably high, e.g., 90% to 99%.

At stage A, a tagged nucleotide (one of four different types: A, T, G, or C) is not associated with the polymerase. At stage B, a tagged nucleotide is associated with the polymerase. At stage C, the polymerase is docked to the nanopore. The tag is pulled into the nanopore during docking by an electrical force, such as a force generated in the presence of an electric field generated by a voltage applied across the membrane and/or the nanopore.

Some of the associated tagged nucleotides are not base paired with the nucleic acid molecule. These non-paired nucleotides typically are rejected by the polymerase within a time scale that is shorter than the time scale for which correctly paired nucleotides remain associated with the polymerase. Since the non-paired nucleotides are only transiently associated with the polymerase, process 400 as shown in FIG. 4 typically does not proceed beyond stage D. For example, a non-paired nucleotide is rejected by the polymerase at stage B or shortly after the process enters stage C.

Before the polymerase is docked to the nanopore, the conductance of the nanopore is −300 picosiemens (300 pS). At stage C, the conductance of the nanopore is about 60 pS, 80 pS, 100 pS, or 120 pS, corresponding to one of the four types of tagged nucleotides respectively. The polymerase undergoes an isomerization and a transphosphorylation reaction to incorporate the nucleotide into the growing nucleic acid molecule and release the tag molecule. In particular, as the tag is held in the nanopore, a unique conductance signal (e.g., see signal 210 in FIG. 2) is generated due to the tag's distinct chemical structures, thereby identifying the added base electronically. Repeating the cycle (i.e., stage A through E or stage A through F) allows for the sequencing of the nucleic acid molecule. At stage D, the released tag passes through the nanopore.

In some cases, tagged nucleotides that are not incorporated into the growing nucleic acid molecule will also pass through the nanopore, as seen in stage F of FIG. 4. The unincorporated nucleotide can be detected by the nanopore in some instances, but the method provides a means for distinguishing between an incorporated nucleotide and an unincorporated nucleotide based at least in part on the time for which the nucleotide is detected in the nanopore. Tags bound to unincorporated nucleotides pass through the nanopore quickly and are detected for a short period of time (e.g., less than 10 ms), while tags bound to incorporated nucleotides are loaded into the nanopore and detected for a long period of time (e.g., at least 10 ms).

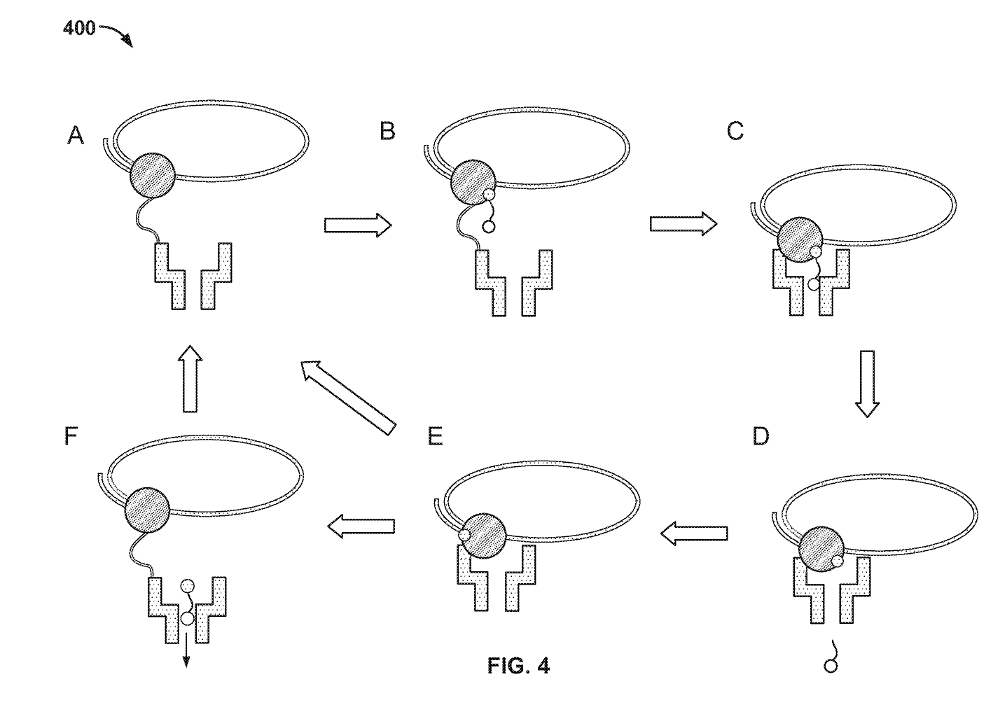

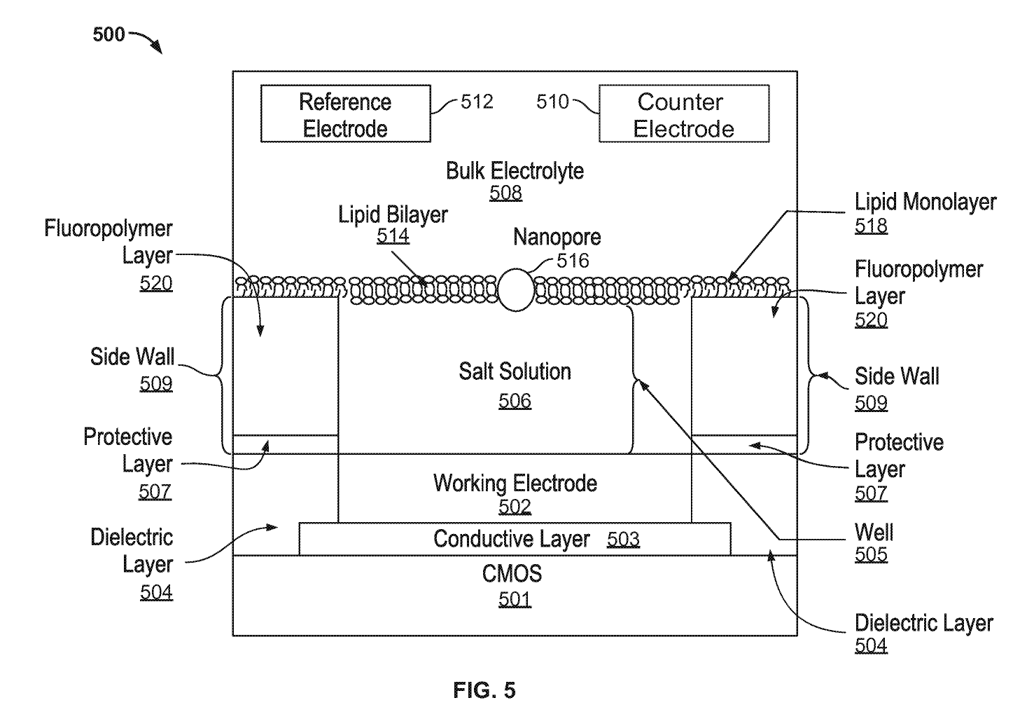

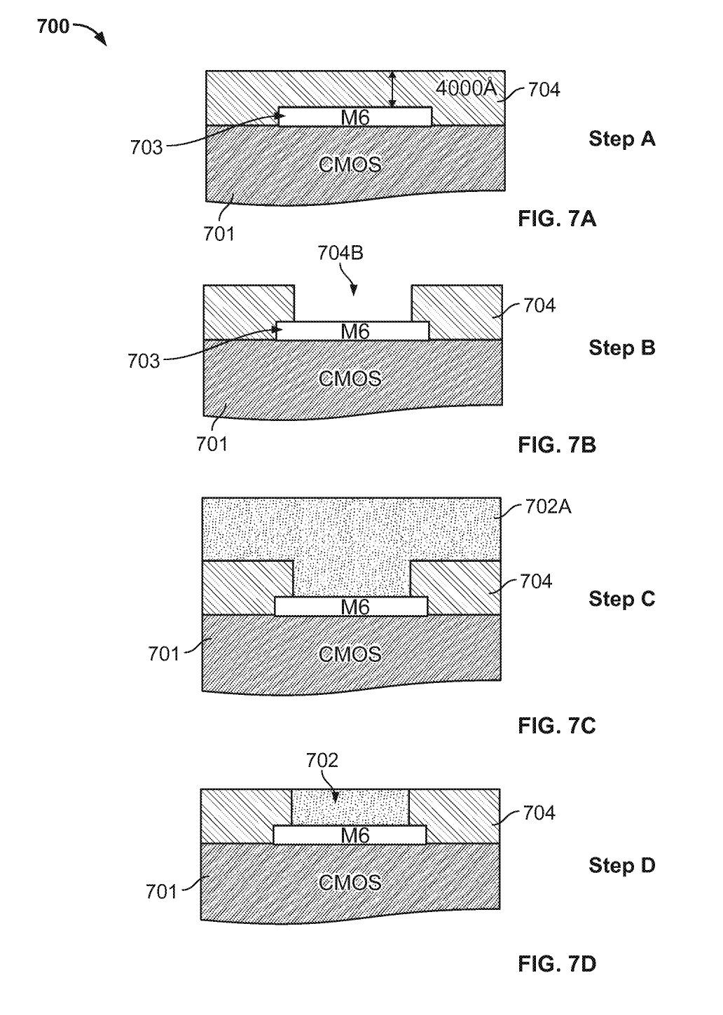

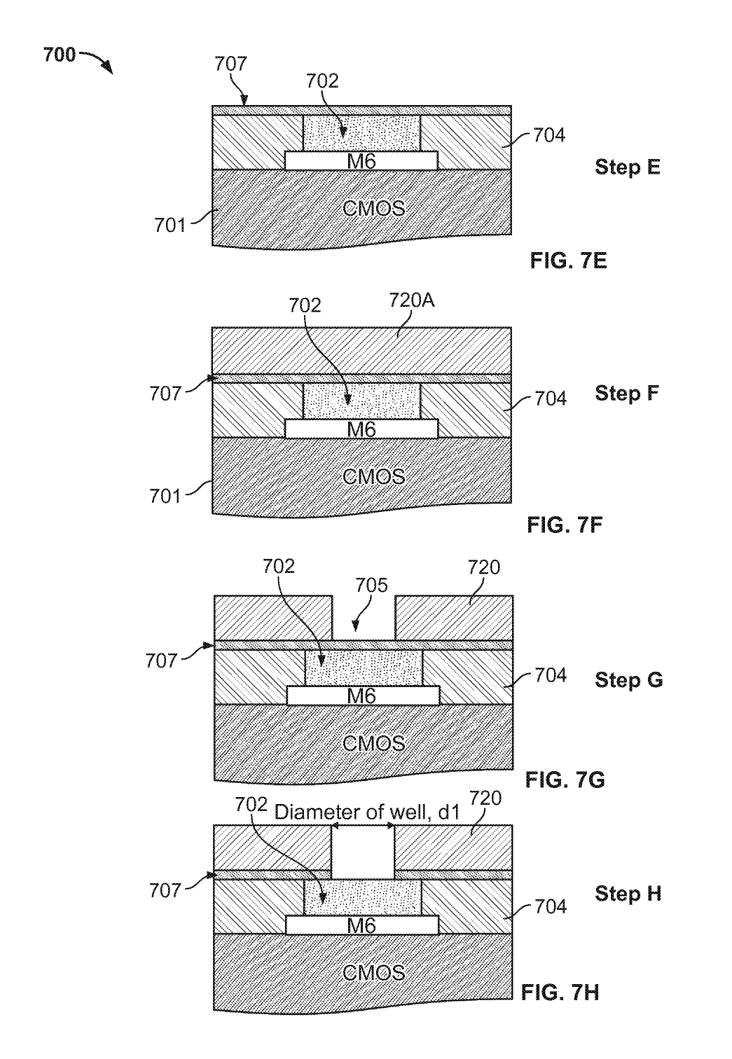

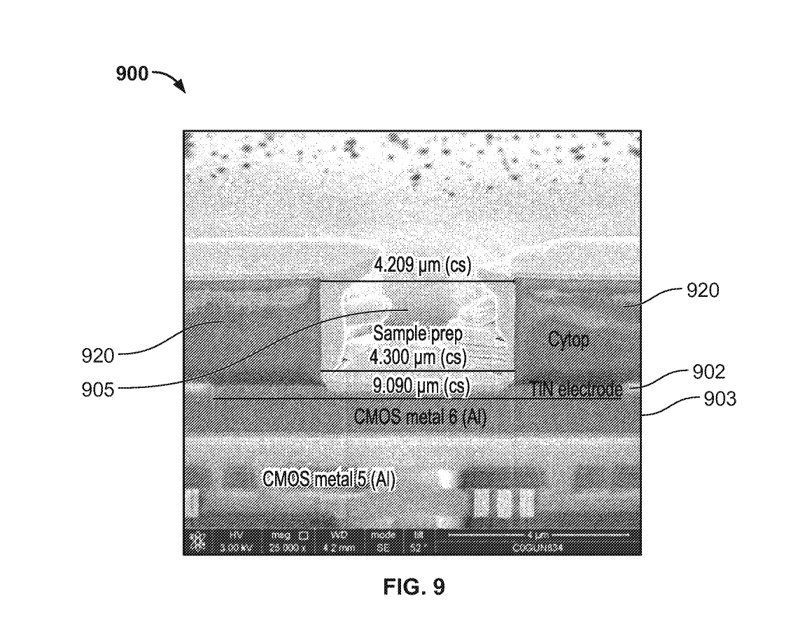

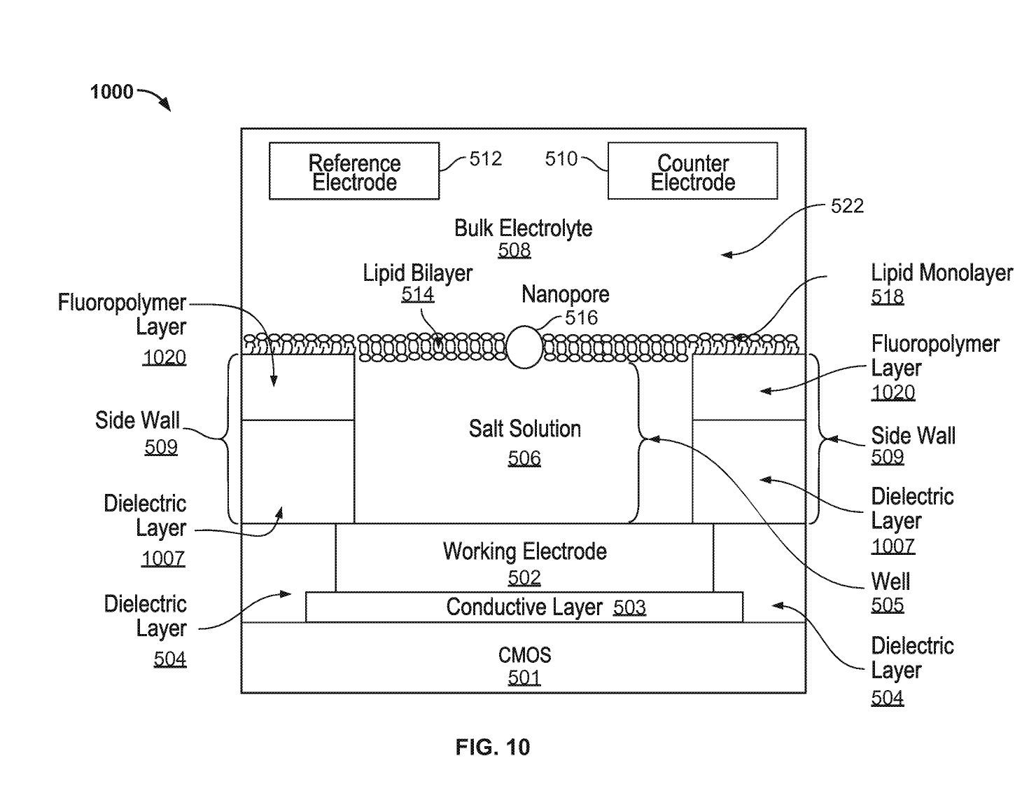

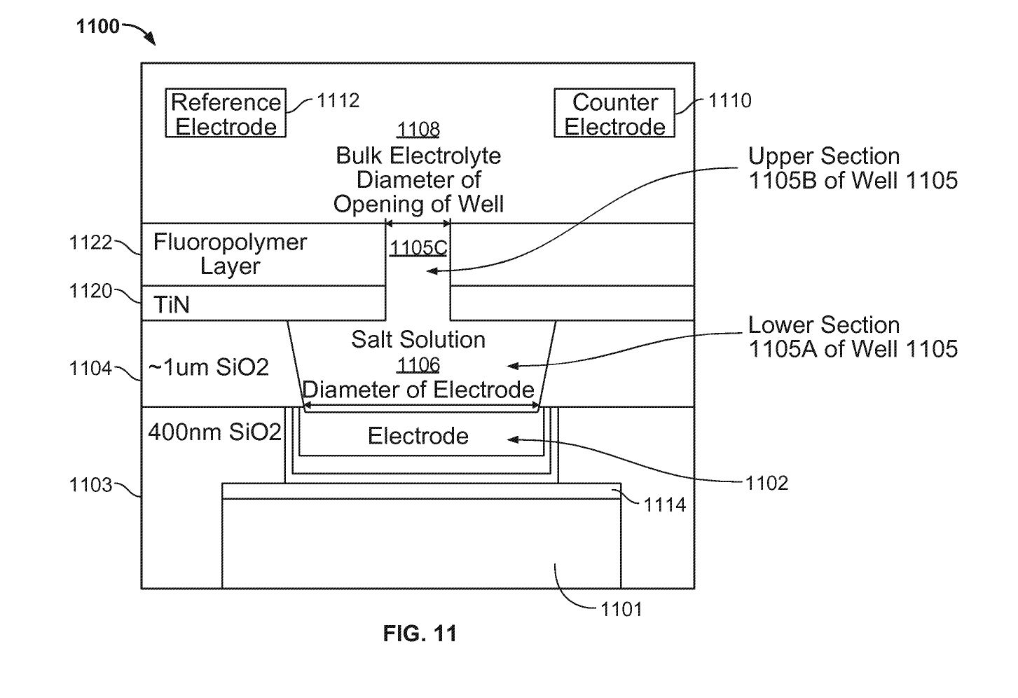

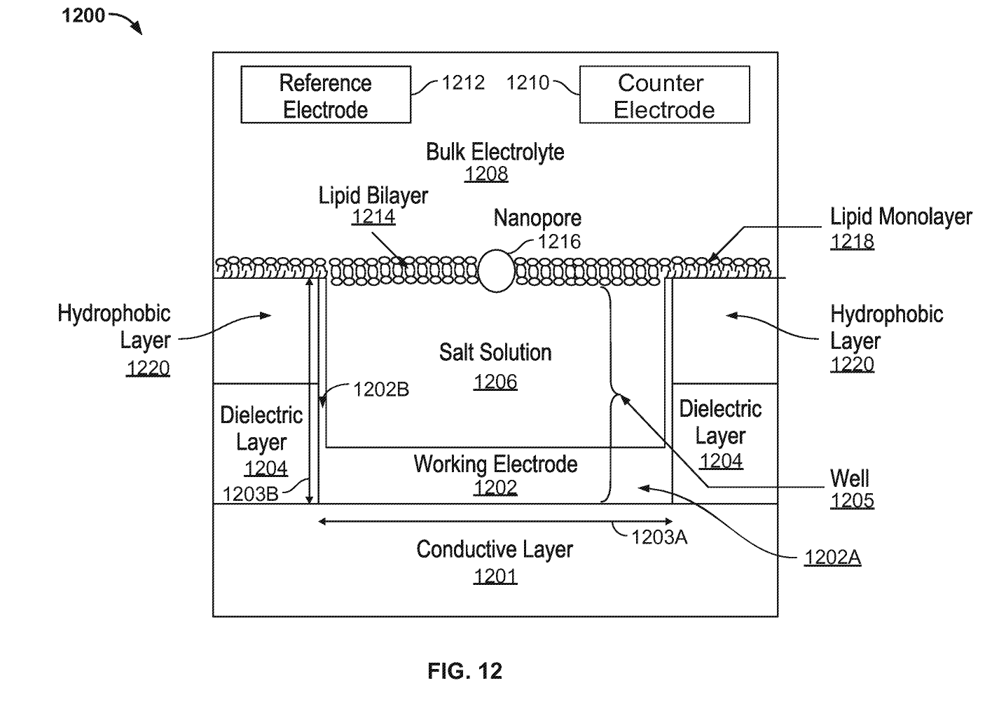

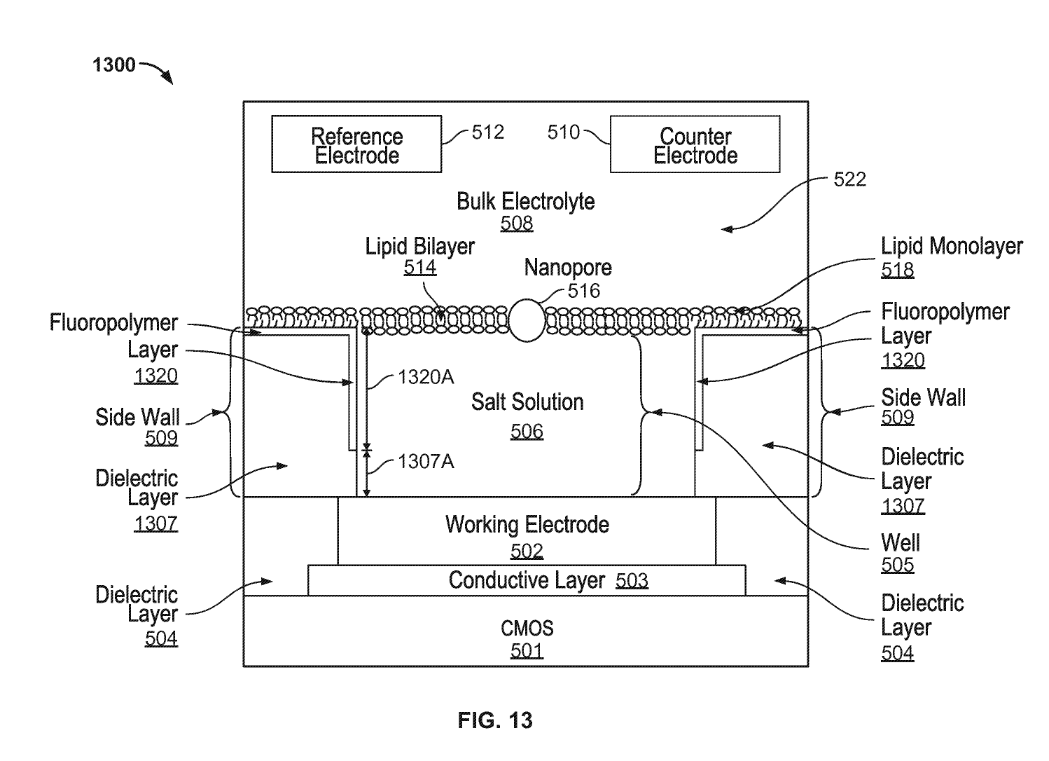

As shown in FIG. 1, a lipid bilayer is formed over the surface of the cell. In particular, each of the cells includes a sensor well with an electrode at the bottom of the well, and a lipid bilayer is formed over the sensor well. In order to facilitate the forming of lipid bilayers over sensor wells, a hydrophobic surface between the wells is desirable. One technique to form a hydrophobic surface is by silanization of a SiO₂ (silicon dioxide) film. In the present application, fluoropolymers are used to form a hydrophobic layer to support lipid bilayer formation for nanopore-based DNA sequencing. A fluoropolymer is a fluorocarbon-based polymer with multiple strong carbon-fluorine bonds. Examples of fluoropolymers that can be used to form the hydrophobic layer include, but are not limited to, Cytop™ and Teflon™ AF. Cytop™ and Teflon™ AF are hereinafter referred to as Cytop and Teflon, respectively. In one aspect, the fluoropolymer-based hydrophobic layer (e.g., fluoropolymer layer 520 in FIG. 5, fluoropolymer layer 720A or 720 in FIG. 7, fluoropolymer hydrophobic side wall 920 in FIG. 9, fluoropolymer layer 1020 in FIG. 10, fluoropolymer layer 1122 in FIG. 11, fluoropolymer hydrophobic layer 1220 in FIG. 12, or fluoropolymer layer 1320 in FIG. 13) is of a suitable thickness. In one embodiment, the thickness of this hydrophobic layer is provided in different units of measurement including, without limitation, an angstrom (Å), a nanometer (nm), or a micron. In one other embodiment, the hydrophobic layer is (i) between about 10 Å and about 20 microns, (ii) between about 30 Å and about 10 microns, (iii) between about 40 Å and about 7 microns, (iv) between about 50 Å and about 5 microns, (v) between about 60 Å and 3 microns, or (vi) between about 80 Å and 1 micron. In other embodiments, the layer is about 10 Å, about 20 Å, about 30 Å, about 40 Å, about 50 Å, about 60 Å, about 70 Å, about 80 Å, about 90 Å, or about 100 Å. In an additional embodiment, the thickness is between about 0.2 microns and about 100 microns. In one embodiment, the thickness is about 0.2 microns, about 0.3 microns, about 0.4 microns, about 0.5 microns, about 0.6 microns, about 0.7 microns, about 0.8 microns, about 0.9 microns, about 1 micron, about 2 microns, about 3 microns, about 4 microns, about 5 microns, about 6 microns, about 7 microns, about 8 microns, about 9 microns, about 10 microns, about 15 microns, about 20 microns, about 25 microns, about 30 microns, about 35 microns, about 40 microns, about 45 microns, about 50 microns, about 55 microns, about 60 microns, about 65 microns, about 70 microns, about 75 microns, about 80 microns, about 85 microns, about 90 microns, about 95 microns, or about 100 microns. In other embodiments, the thickness is (i) between about 0.5 nm and about 1000 nm, or (ii) between about 1 nm and 100 nm. In another embodiment, the thickness of the fluoropolymer layer is about 0.5 nm, about 1 nm, about 1.5 nm, about 2 nm, about 2.5 nm, about 3 nm, about 4 nm, about 5 nm, about 6 nm, about 7 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, or about 1000 nm.

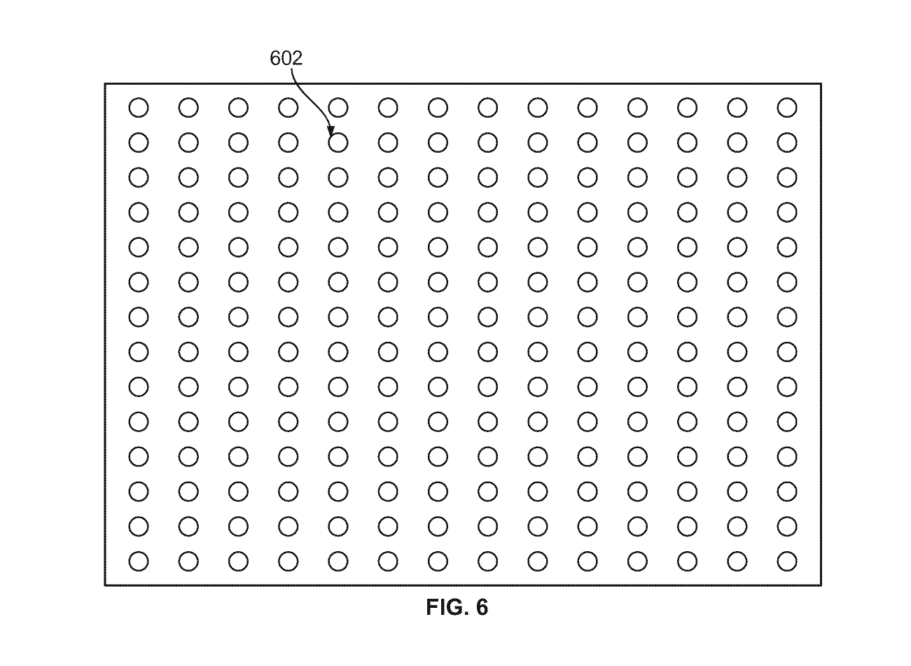

FIG. 5 illustrates a cross-sectional view of an embodiment of an electrochemical cell 500 in a nanopore-based sequencing chip that includes a fluoropolymer hydrophobic layer to facilitate the formation of a lipid bilayer. Cell 500 includes a well 505 having a side wall 509 and a bottom. The bottom of well 505 comprises a working electrode 502. Well 505 has an opening above an uncovered portion of working electrode 502. In some embodiments, the opening above the uncovered portion of the working electrode is circular or octagonal in shape. FIG. 6 illustrates a top view of a plurality of circular openings 602 of a plurality of wells in a nanopore-based sequencing chip. In some embodiments, well 505 can accommodate (comprises, or can comprise) a volume of between about 1 attoliter and about 1 nanoliter.

In some embodiments, working electrode 502 is a metal electrode. In some embodiments, working electrode 502 is circular or octagonal in shape and a dielectric layer 504 forms the walls surrounding working electrode 502. For non-faradaic conduction, working electrode 502 may be made of metals that are resistant to corrosion and oxidation, e.g., platinum, gold, titanium nitride and graphite. For example, working electrode 502 may be a platinum electrode with electroplated platinum. In another example, working electrode 502 may be a titanium nitride (TiN) working electrode. The electrochemical capacitance associated with a TiN working electrode may be increased by maximizing the specific surface area of the electrode. The specific surface area of working electrode 502 is the total surface area of the electrode per unit of mass (e.g., m²/kg) or per unit of volume (e.g., m²/m³ or m⁻¹) or per unit of base area (e.g., m²/m²) As the surface area increases, the electrochemical capacitance of the working electrode increases, and a greater amount of ions can be displaced with the same applied potential before the capacitor becomes charged. The surface area of working electrode 502 may be increased by making the TiN electrode “spongy” or porous, with many sparsely-spaced columnar structures of TiN therein.

Working electrode 502 has a top side and a bottom side. The top side of working electrode 502 makes up the bottom of well 505 while the bottom side of working electrode 502 is in contact with a conductive or metal layer 503. Conductive layer 503 connects cell 500 to the remaining portions of the nanopore-based sequencing chip. In some embodiments, conductive layer 503 is on top of a CMOS base 501.

In some embodiments, dielectric layer 504 forms the walls surrounding working electrode 502. In some embodiments, the side wall 509 of well 505 is above dielectric layer 504. Suitable dielectric materials for use in the present invention (e.g., as shown in FIG. 5 for dielectric layer 504 and in FIG. 10 for dielectric layer 1007) include, without limitation, porcelain (ceramic), glass, mica, plastics, oxides, nitrides (e.g., silicon mononitride or SiN, as well as silicon nitride or Si₃N₄), silicon oxynitride, metal oxides, metal nitrides, metal silicates, transition-metal oxides, transition-metal nitrides, transition metal-silicates, oxynitrides of metals, metal aluminates, zirconium silicate, zirconium aluminate, hafnium oxide, insulating materials (e.g., polymers, epoxies, photoresist, and the like), or combinations thereof. Those of ordinary skill in the art will appreciate that other dielectric materials are suitable for use in the present invention.

Well 505 further includes a volume of salt solution 506 above working electrode 502. In general, different solutions in cell 500 (e.g., salt solution 506 or bulk electrolyte 508) comprise osmolytes. As used herein, the term “osmolyte” refers to any soluble compound that when dissolved into solution increases the osmolarity of that solution. Osmolytes for use in the present invention include, without limitation, ionic salts such as lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl₂), strontium chloride (SrCl₂), manganese chloride (MnCl₂), and magnesium chloride (MgCl₂); polyols and sugars such as glycerol, erythritol, arabitol, sorbitol, mannitol, xylitol, mannisidomannitol, glycosyl glycerol, glucose, fructose, sucrose, trehalose, and isofluoroside; polymers such as dextrans, levans, and polyethylene glycol; and some amino acids and derivatives thereof, such as glycine, alanine, alpha-alanine, arginine, proline, taurine, betaine, octopine, glutamate, sarcosine, y-aminobutyric acid, and trimethylamine N-oxide (“TMAO”) (see also e.g., Fisher et al. U.S. 20110053795, incorporated herein by reference in its entirety).

Cell 500 includes a counter electrode (CE) 510 which is in electrical contact with a bulk electrolyte 508. Cell 500 may optionally include a reference electrode 512. In some embodiments, counter electrode 510 is shared between a plurality of cells and is therefore also referred to as a common electrode. The common electrode can be configured to apply a common potential to the bulk liquid in contact with the nanopores in the measurements cells. The common potential and the common electrode are common to all of the measurement cells.

As shown in FIG. 5, a membrane is formed on the top surfaces of side wall 509 of well 505 and spans across well 505. For example, the membrane includes a lipid monolayer 518 formed on top of side wall 509. As the membrane reaches the opening of well 505, the lipid monolayer transitions to a lipid bilayer 514 that spans across the opening of the well. Bulk electrolyte 508 containing protein nanopore transmembrane molecular complexes (PNTMC) and the analyte of interest is placed directly above the well. A single PNTMC/nanopore 516 is inserted into lipid bilayer 514. In one embodiment, insertion into the bilayer is by electroporation. Nanopore 516 crosses lipid bilayer 514 and provides the only path for ionic flow from bulk electrolyte 508 to working electrode 502. An electrolyte solution is present both inside well 505, i.e., trans side, (see salt solution 506) and in a much larger external reservoir 522, i.e., cis side, (see bulk electrolyte 508). The bulk electrolyte 508 in external reservoir 522 is above multiple wells of the nanopore-based sequencing chip. Lipid bilayer 514 extends over well 505 and transitions to lipid monolayer 518 where the monolayer is attached to the top surfaces of side wall 509. This geometry both electrically and physically seals well 505 and separates the well from the larger external reservoir. While neutral molecules, such as water and dissolved gases, may pass through lipid bilayer 514, ions may not. Nanopore 516 in lipid bilayer 514 provides a single path for ions to be conducted into and out of well 505.

For nucleic acid sequencing, a polymerase is attached to nanopore 516. A template of nucleic acid (e.g., DNA) is held by the polymerase. For example, the polymerase synthesizes DNA by incorporating hexaphosphate mono-nucleotides (HMN) from solution that are complementary to the template. A unique, polymeric tag is attached to each HMN. During incorporation, the tag threads the nanopore aided by an electric field gradient produced by the voltage between counter electrode 510 and working electrode 502. The tag partially blocks nanopore 516, procuring a measurable change in the ionic current through nanopore 516. In some embodiments, an alternating current (AC) bias or a direct current (DC) voltage is applied between the electrodes.

In order to facilitate the forming of lipid bilayers over sensor wells, side wall 509 comprises a fluoropolymer layer 520. A fluoropolymer is a fluorocarbon-based polymer with multiple strong carbon-fluorine bonds. Examples of fluoropolymer that can be used to form the hydrophobic layer include, but are not limited to, Cytop™ and Teflon™ AF.

Fluoropolymer layer 520 provides a top surface and a vertical surface that are hydrophobic, which facilitate the adhesion of a membrane (e.g., a lipid bilayer comprising a nanopore) and the transition of the membrane from a lipid monolayer to a lipid bilayer. The membrane above cell 500 includes lipid monolayer 518 formed on top of the top surface of fluoropolymer layer 520. As the membrane reaches the opening of well 505, the lipid monolayer transitions to lipid bilayer 514 that spans across the opening of the well. Lipid monolayer 518 may also extend along all or a part of the vertical surface of side wall 509, which is all or a part of the vertical surface of fluoropolymer layer 520.

Fluoropolymer layer 520 forms the side wall 509 surrounding well 505 in which a working electrode 502 is located at the bottom. In some embodiments, fluoropolymer layer 520 has a thickness between one and ten microns. In some embodiments, the bottom of side wall 509 comprises a thin protective layer 507. In one example, protective layer 507 is formed using SiO₂ (silicon dioxide). In one aspect, the present invention provides a protective layer (e.g., protective layer 507 in FIG. 5, or protective layer 707 in FIG. 7) deposited on top of the working electrode. In one embodiment, the protective layer is between the fluoropolymer layer and the working electrode. In one other embodiment, the protective layer comprises silicon dioxide (SiO₂). In another embodiment, the protective layer is of a suitable thickness. In other embodiments, the thickness of this protective layer is provided in different units of measurement including, without limitation, an angstrom (Å), a nanometer (nm), or a micron. In one other embodiment, the protective layer is (i) between about 10 Å and about 20 microns, (ii) between about 30 Å and about 10 microns, (iii) between about 40 Å and about 7 microns, (iv) between about 50 Å and 5 microns, (v) between about 60 Å and 3 microns, (vi) between about 80 Å and 1 micron, or (vii) between about 10 Å and about 300 Å. In other embodiments, the protective layer has a thickness of about 10 Å, about 20 Å, about 30 Å, about 40 Å, about 50 Å, about 60 Å, about 70 Å, about 80 Å, about 90 Å, about 100 Å, about 200 Å, about 300 Å, about 400 Å, about 500 Å, about 600 Å about 700 Å, about 800 Å, about 900 Å, or about 1000 Å. In one other embodiment, the thickness of the protective layer is between about 10 nm and about 1000 nm. In another embodiment, the thickness is about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, or about 1000 nm. In an additional embodiment, the thickness of the protective layer is between about 0.2 microns and about 100 microns. In one embodiment, the thickness of the protective layer is about 0.2 microns, about 0.3 microns, about 0.4 microns, about 0.5 microns, about 0.6 microns, about 0.7 microns, about 0.8 microns, about 0.9 microns, about 1 micron, about 2 microns, about 3 microns, about 4 microns, about 5 microns, about 6 microns, about 7 microns, about 8 microns, about 9 microns, about 10 microns, about 20 microns, about 30 microns, about 35 microns, about 40 microns, about 45 microns, about 50 microns, about 55 microns, about 60 microns, about 65 microns, about 70 microns, about 75 microns, about 80 microns, about 85 microns, about 90 microns, about 95 microns, or about 100 microns.

In cell 500, the base surface area of the opening of well 505 (which is the same as the base surface area of lipid bilayer 514) and the base surface area of working electrode 502 are determined by the dimensions of side wall 509 and dielectric layer 504, respectively. The base surface area of working electrode 502 is greater than or equal to the base surface area of the opening of well 505.

FIGS. 7A-7H illustrate the various steps of an embodiment of a process 700 for constructing a non-faradaic electrochemical cell of a nanopore-based sequencing chip that includes a fluoropolymer hydrophobic layer to facilitate the formation of a lipid bilayer and a TiN working electrode with increased electrochemical capacitance.

At step A, a layer of dielectric 704 (e.g., SiO₂) is disposed on top of a conductive layer 703 (e.g, M6) and a CMOS base 701. Conductive layer 703 includes circuitry that delivers the signals from the cell to the rest of the chip. For example, the circuitry delivers signals from the cell to an integrating capacitor. In some embodiments, the layer of dielectric 704 has a thickness of about 4000 Å (one angstrom, Å, is 10⁻¹⁰ meter) on top of conductive layer 703.

At step B, the layer of dielectric 704 is etched to create a hole 704B. The hole 704B exposes the top surface of conductive layer 703 and provides a space for growing a spongy and porous TiN electrode.

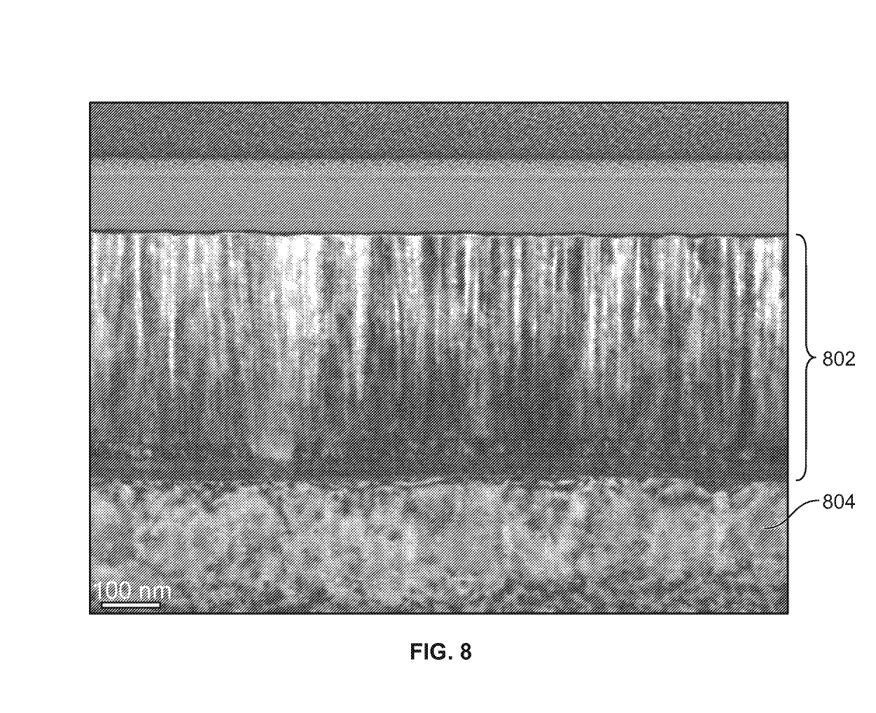

At step C, a spongy and porous TiN layer 702A is deposited to fill the hole 704B created at step B. The spongy and porous TiN layer 702A is grown and deposited in a manner to create rough, sparsely-spaced TiN columnar structures or columns of TiN crystals that provide a high specific surface area which can come in contact with an electrolyte. The layer of spongy and porous TiN layer 702A can be deposited using different deposition techniques, including atomic layer deposition, chemical vapor deposition, physical vapor deposition (PVD) sputtering deposition, and the like. For example, layer 702A may be deposited by chemical vapor deposition using TiCl₄ in combination with nitrogen containing precursors (e.g., NH₃ or N₂). Layer 702A may also be deposited by chemical vapor deposition using TiCl₄ in combination with titanium and nitrogen containing precursors (e.g., tetrakis-(dimethylamido) titanium (TDMAT) or tetrakis-(diethylamido) titanium TDEAT). Layer 702A may also be deposited by PVD sputtering deposition. For example, titanium can be reactively sputtered in an N₂ environment or directly sputtered from a TiN target. The conditions of each of the deposition methods may be tuned in such a way to deposit sparsely-spaced TiN columnar structures or columns of TiN crystals. For example, when layer 702A is deposited by DC (direct current) reactive magnetron sputtering from a titanium (Ti) target, the deposition system can be tuned to use a low temperature, low substrate bias voltage (the DC voltage between the silicon substrate and the Ti target) and high pressure (e.g., 25 mT), such that the TiN can be deposited more slowly and more gently to form columns of TiN crystals. In some embodiments, the depth of the deposited layer 702A is about 1.5 times the depth of hole 704B. The depth of the deposited layer 702A is between 500 angstroms to 3 microns thick. The diameter or width of the deposited layer 702A is between 20 nm to 100 microns. FIG. 8 illustrates a cross-section view of a spongy and porous TiN layer 802 deposited above a metal layer 804. As shown in FIG. 8, the spongy and porous TiN layer 802 includes grass-like columnar structures.

With continued reference to FIG. 7D, at step D, the excess TiN layer is removed. For example, the excess TiN layer may be removed using chemical mechanical polishing (CMP) techniques. The remaining TiN deposited in the hole 704B forms a spongy and porous TiN working electrode 702.

At step E, after working electrode 702 is formed, a protective layer 707 is deposited on top of working electrode 702 and dielectric 704. In one example, protective layer 707 is formed using SiO₂ (silicon dioxide). In some embodiments, a protective layer having a suitable thickness (as described herein) is formed. In another embodiment, the protective layer 707 has a thickness of between about 10 angstroms and about 50 microns.

At step F, a fluoropolymer hydrophobic layer 720A (e.g, a Cytop layer) is deposited on top of the protective layer 707. For example, a Cytop layer is spun on using a track. In some embodiments, a fluoropolymer layer 720A having a suitable thickness (as described herein) is deposited. In one embodiment, the thickness of fluoropolymer layer 720A is between about 0.5 microns and about 6 microns.

At step G, fluoropolymer layer 720A is etched to create a well 705 exposing a portion of the upper surface of protective layer 707. For example, the well may be etched using a fluorine based plasma.

At step H, the exposed portion of protective layer 707 is etched to expose a portion of the upper surface of working electrode 702. For example, reactive-ion etching (RIE) may be used. In some embodiments, the diameter (dl) of well 705 is between 20 nm to 100 microns.

FIG. 9 illustrates a cross-sectional photograph of an electrochemical cell 900 in a nanopore-based sequencing chip that includes a fluoropolymer hydrophobic layer to facilitate the formation of a lipid bilayer. Electrochemical cell 900 includes a working electrode 902 above a conductive layer 903. Electrochemical cell 900 includes a well 905 having a fluoropolymer hydrophobic side wall 920 and a bottom. The bottom of well 905 comprises working electrode 902. As shown in FIG. 9, the top surface and vertical surface of fluoropolymer side wall 920 and the top surface of working electrode 902 are covered by a sample preparation material.

FIG. 10 illustrates a cross-sectional view of another embodiment of an electrochemical cell 1000 in a nanopore-based sequencing chip that includes a fluoropolymer hydrophobic layer to facilitate the formation of a lipid bilayer. Electrochemical cell 1000 and electrochemical cell 500 (see FIG. 5) share a number of identical parts (as indicated by identical numerals in FIGS. 5 and 10), including dielectric layer 504, CMOS 501, conductive layer 503, working electrode 502, salt solution 506, side wall 509, well 505, lipid monolayer 518, lipid bilayer 514, nanopore 516, bulk electrolyte 508, counter electrode 510, reference electrode 512, and external reservoir 522.

One difference between cell 500 and cell 1000 is the composition of the side wall 509. In cell 500, side wall 509 comprises a fluoropolymer layer 520 and a thin protective layer 507 at the base of the side wall. In cell 1000, a fluoropolymer hydrophobic layer 1020 and a dielectric layer 1007 together form the insulating side wall 509 surrounding well 505. In particular, the upper portion of side wall 509 is a fluoropolymer hydrophobic layer 1020 and the bottom portion of side wall 509 is a dielectric layer 1007, such that the top horizontal surface of side wall 509 and the upper vertical surface of side wall 509 are hydrophobic, while the lower vertical surface of side wall 509 is either hydrophilic or hydrophobic. In one embodiment, the dielectric layer 1007 comprises SiO₂ which is generally hydrophilic in an unmodified state. In another embodiment, the dielectric layer 1007 comprises a surface that forms a sidewall of well 505. In one embodiment, the sidewall surface comprises SiO₂ modified to render the surface hydrophobic in nature. For instance, hydrophobic groups can be chemically bonded to SiO₂ on the surface forming a sidewall of well 505. In one embodiment, the hydrophobic groups include, without limitation, alkyl or polydimethylsiloxane chains. The hydrophobic top surface and hydrophobic vertical surface provided by fluoropolymer layer 1020 facilitate the adhesion of a membrane (e.g., a lipid bilayer comprising a nanopore) and the transition of the membrane from a lipid monolayer to a lipid bilayer. In some embodiments, the combined thickness of fluoropolymer layer 1020 and dielectric layer 1007 is provided as a suitable thickness (as described herein). In one embodiment, the thickness of fluoropolymer layer 1020 is between about 100 nanometers (nm) and about 10 microns. In some embodiments, dielectric layer 1007 is formed using silicon dioxide (SiO₂). However, other materials may be used to form dielectric layer 1007, as described herein.

FIG. 11 illustrates a cross-sectional view of another embodiment of an electrochemical cell 1100 in a nanopore-based sequencing chip that includes a fluoropolymer hydrophobic layer to facilitate the formation of a lipid bilayer. Cell 1100 has a smaller aperture opening to a chalice well for the formation of a lipid bilayer with a smaller base surface area and a working electrode with a larger base surface area. The base surface area of the opening to the well (which is the same as the base surface area of the lipid bilayer) and the top base surface area of the working electrode that is exposed to the electrolyte can be adjusted independently of each other.

Cell 1100 includes a conductive or metal layer 1101. Metal layer 1101 connects cell 1100 to the remaining portions of the nanopore-based sequencing chip. In some embodiments, metal layer 1101 is the metal 6 layer (M6). Cell 1100 further includes a working electrode 1102 and a dielectric layer 1103 above metal layer 1201. In some embodiments, the base surface area of working electrode 1102 is circular or octagonal in shape and dielectric layer 1103 forms the walls surrounding working electrode 1102. Cell 1100 further includes a dielectric layer 1104 above working electrode 1102 and dielectric layer 1103. Dielectric layer 1104 forms the insulating side wall surrounding a lower section (1105A) of a well 1105. In some embodiments, dielectric layer 1103 and dielectric layer 1104 together form a single piece of dielectric. Dielectric layer 1103 is the portion that is disposed horizontally adjacent to working electrode 1102, and dielectric layer 1104 is the portion that is disposed above the working electrode. In some embodiments, dielectric layer 1103 and dielectric layer 1104 are separate pieces of dielectric and they may be grown separately. Dielectric material used to form dielectric layers 1103 and 1104 includes glass, oxide, silicon mononitride (SiN), silicon nitride (Si₃N₄), silicon dioxide (SiO₂), and the like.

Cell 1100 further includes a hydrophilic layer 1120 (e.g., titanium nitride, TiN) and a hydrophobic layer 1122 above dielectric layer 1104. Hydrophilic layer 1120 and hydrophobic layer 1122 together form the insulating side wall surrounding an upper section (1105B) of well 1105. Hydrophilic layer 1120 and hydrophobic layer 1122 together form an overhang above the lower section (1105A) of well 1105. Alternatively, hydrophilic layer 1120 is optional. Hydrophobic layer 1122 forms the insulating wall surrounding upper section 1105B of well 1105. Hydrophobic layer 1122 forms an overhang above the lower section (1105A) of well 1105. Hydrophobic layer 1122 is formed with a fluoropolymer, such as Cytop and Teflon. In some embodiments, hydrophobic layer 1122 has an appropriate thickness (as described herein). In another embodiment, the thickness of hydrophobic layer 1122 is between about 100 angstroms and 2 microns. The interface between hydrophobic layer 1122 and hydrophilic layer 1120 facilitates the formation of a stable lipid bilayer. The lipid bilayer is formed at the interface between hydrophobic layer 1122 and hydrophilic layer 1120.

The upper section 1105B of well 1105 has an opening 1105C above the working electrode. In some embodiments, opening 1105C above the working electrode is circular and the base surface area of the opening is π×(d/2)², where d is the diameter of the opening. In some embodiments, opening 1105C above the working electrode is octagonal in shape. The base surface areas of opening 1105C and the upper section 1105B of well 1105, respectively, are smaller than the bottom base surface area of the lower section 1105A of well 1105. As the lipid bilayer spans across opening 1105C, a reduction in the base surface area of opening 1105C results in a reduction in the base surface area of the lipid bilayer and also the capacitance associated with the lipid bilayer. The lower section 1105A of well 1105 provides a large reservoir/chalice with a bottom base surface area larger than that in the upper section 1105B of well 1105. An increase in the bottom base surface area of the lower section 1105A of well 1105 increases the top base surface area of the electrode that has direct contact with the electrolye/salt solution 1106, thereby increasing the electrochemical capacitance associated with the working electrode.

Inside well 1105, salt solution/electrolyte 1106 is deposited above working electrode 1102. Salt solution 1106 may include one of the following: lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl₂), strontium chloride (SrCl₂), manganese chloride (MnCl₂), and magnesium chloride (MgCl₂). In some embodiments, salt solution 1106 has a thickness of about three microns (μm). The thickness of salt solution 1106 may range from 0 to 5 microns.

A bulk electrolyte 1108 containing protein nanopore transmembrane molecular complexes (PNTMC) and the analyte of interest is placed directly above the well. A single PNTMC/nanopore is inserted into the lipid bilayer by electroporation. The nanopore crosses the lipid bilayer and provides the only path for ionic flow from bulk electrolyte 1108 to working electrode 1102. Bulk electrolyte 1108 may further include one of the following: lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl₂), strontium chloride (SrCl₂), manganese chloride (MnCl₂), and magnesium chloride (MgCl₂).

Cell 1100 includes a counter electrode (CE) 1110. Cell 1100 also includes a reference electrode 1112, which acts as an electrochemical potential sensor. In some embodiments, counter electrode 1110 is shared between a plurality of cells, and is therefore also referred to as a common electrode. The common electrode can be configured to apply a common potential to the bulk liquid in contact with the nanopores in the measurements cells. The common potential and the common electrode are common to all of the measurement cells.

In some embodiments, working electrode 1102 is a titanium nitride (TiN) working electrode with increased electrochemical capacitance. The electrochemical capacitance associated with working electrode 1102 may be increased by maximizing the specific surface area of the electrode. The specific surface area of working electrode 1102 is the total surface area of the electrode per unit of mass (e.g., m²/kg), per unit of volume (e.g., m²/m³ or m⁻¹), or per unit of base area (e.g., m²/m²). As the surface area increases, the electrochemical capacitance of the working electrode increases, and a greater amount of ions can be displaced with the same applied potential before the capacitor becomes charged. The surface area of working electrode 1102 may be increased by making the TiN electrode “spongy” or porous. The TiN sponge soaks up electrolyte and creates a large effective surface area in contact with the electrolyte.

FIG. 12 illustrates a cross-sectional view of another embodiment of an electrochemical cell 1200 in a nanopore-based sequencing chip that includes a fluoropolymer hydrophobic layer to facilitate the formation of a lipid bilayer. Cell 1200 has a bowl-shaped or cup-shaped working electrode that can provide an increased current in the cell. Cell 1200 is one of the cells in a nanopore based sequencing chip.

One difference between cell 1200 and other cells disclosed above (e.g., cell 500, cell 1000, and cell 1100) is the shape and construction of their respective working electrodes. The working electrodes in cell 500, cell 1000, and cell 1100 are planar electrodes located at the bottom of a well. Working electrode 1202 of cell 1200 is a bowl-shaped electrode; it can also be lidless and box-shaped, cup-shaped or bucket-shaped. The bowl-shaped working electrode 1202 has a planar portion 1202A at the bottom, forming the base of the bowl. The base surface area may be circular or octagonal in shape. The bowl-shaped working electrode 1202 further includes a surrounding wall 1202B extending perpendicular to (or at an angle from) the planar portion and along the periphery of the planar portion. Both the upper surface of the planar portion 1202A and the interior surface of the surrounding wall 1202B provide an electrode surface area that is exposed to the electrolyte 1206. The surrounding wall 1202B takes advantage of the vertical device real estate, i.e., the space orthogonal to the substrate plane. The width (or diameter) of the planar portion 1202A is indicated by 1203A of FIG. 12, and the height of the surrounding wall 1202B is indicated by 1203B of FIG. 12. In some embodiments, width 1203A is between 1 to 100 microns, and height 1203B is between 100 nm to 20 microns. In one embodiment, width 1203A is about 5.5 microns and height 1203B is about 3.5 microns. The ratio between 1203B and 1203A is referred to as the aspect ratio of working electrode 1202. The aspect ratio may be less than or greater than one.

Working electrode 1202 can provide an increased current in cell 1200 as compared to planar working electrodes. In some embodiments, the current that can be provided to cell 1200 may be tuned by adjusting the aspect ratio of working electrode 1202.

Working electrode 1202 can also provide an increased capacitance. Both the upper surface of planar portion 1202A and the interior surface of surrounding wall 1202B of electrode 1202 provide an electrode surface area that is exposed to the electrolyte 1206, thereby increasing the capacitance associated with working electrode 1202.

In cell 1200, a fluoropolymer hydrophobic layer 1220 provides a hydrophobic top surface to facilitate the adhesion of a membrane (e.g., a lipid bilayer comprising a nanopore) and the transition of the membrane from a lipid monolayer to a lipid bilayer. Cell 1200 may include an optional dielectric layer 1204, and the fluoropolymer layer 1220 is positioned above the dielectric layer 1204. In some embodiments, the combined thickness of fluoropolymer layer 1220 and dielectric layer 1204 is between one to ten microns. The thickness of fluoropolymer layer 1220 is between 100 nm to 10 microns. In some embodiments, dielectric layer 1204 is formed using silicon dioxide (SiO₂). However, other materials may be used to form dielectric layer 1204, as described herein.

FIG. 13 illustrates a cross-sectional view of another embodiment of an electrochemical cell 1300 in a nanopore-based sequencing chip that includes a fluoropolymer hydrophobic layer to facilitate the formation of a lipid bilayer. Electrochemical cell 1300 and electrochemical cell 500 (see FIG. 5) share a number of identical parts (as indicated by identical numerals in FIGS. 5 and 13), including dielectric layer 504, CMOS 501, conductive layer 503, working electrode 502, salt solution 506, side wall 509, well 505, lipid monolayer 518, lipid bilayer 514, nanopore 516, bulk electrolyte 508, counter electrode 510, reference electrode 512, and external reservoir 522.

One difference between cell 500 and cell 1300 is the composition of the side wall 509. In cell 500, side wall 509 comprises a fluoropolymer layer 520 and a thin protective layer 507 at the base of the side wall. In cell 1300, a fluoropolymer hydrophobic layer 1320 and a dielectric layer 1307 together form the insulating side wall 509 surrounding well 505. In particular, the top horizontal surface of side wall 509 and an upper portion of the vertical surface of side wall 509 are covered by a fluoropolymer hydrophobic layer 1320 and the lower portion of the vertical surface of side wall 509 is a dielectric layer 1307, such that the top horizontal surface of side wall 509 and the upper vertical surface of side wall 509 are hydrophobic, while the lower vertical surface of side wall 509 is not hydrophobic. The hydrophobic top surface and hydrophobic vertical surface provided by fluoropolymer layer 1320 facilitate the adhesion of a membrane (e.g., a lipid bilayer comprising a nanopore) and the transition of the membrane from a lipid monolayer to a lipid bilayer. The thickness of the upper vertical surface of side wall 509 that is covered by fluoropolymer layer 1320 is indicated as 1320A. The thickness of the lower vertical surface of side wall 509 that is a dielectric surface is indicated as 1307A. In some embodiments, the combined thickness of 1320A and 1307A is between one to ten microns. The thickness of the fluoropolymer on the top surface of the side wall is provided at an appropriate thickness (as described herein). In one other embodiment, the thickness of the fluoropolymer layer on the top surface of the side wall is between about 100 nm and about 50 microns. In some embodiments, dielectric layer 1307 is formed using silicon dioxide (SiO₂). However, other materials may be used to form dielectric layer 1307, as described herein.

Although the foregoing embodiments have been described in some detail for purposes of clarity of understanding, the invention is not limited to the details provided. There are many alternative ways of implementing the invention. The disclosed embodiments are illustrative and not restrictive.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of sequencing a DNA sample, including:
providing a nanopore-based sequencing device, comprising:
a conductive layer;
a working electrode disposed above the conductive layer; and
a side wall disposed above the working electrode, wherein the side wall and the working electrode form a well in which an electrolyte may be contained, and
wherein at least an upper portion of the side wall comprises a hydrophobic portion formed by a fluoropolymer material; and
sequencing the DNA sample using the nanopore-based sequencing device.

2. The method of claim 1, wherein the fluoropolymer material is selected from the group consisting of Cytop and Teflon.

3. The method of claim 1, wherein a thickness of the hydrophobic portion formed by the fluoropolymer material is between about 10 angstroms and about 100 microns.

4. The method of claim 1, wherein the hydrophobic portion formed by the fluoropolymer material comprises a top horizontal hydrophobic surface above the well that facilitates the formation of a lipid bilayer that spans across the well.

5. The method of claim 1, wherein the hydrophobic portion formed by the fluoropolymer material comprises a vertical hydrophobic surface inside the well that facilitates the formation of a lipid bilayer that spans across the well.

6. The method of claim 1, wherein at least a lower portion of the side wall comprises a dielectric layer, and wherein the hydrophobic portion formed by the fluoropolymer material is disposed above the dielectric layer.

7. The method of claim 6, wherein a combined thickness of the hydrophobic portion formed by the fluoropolymer material and the dielectric layer is between one to ten microns.

8. The method of claim 6, wherein the dielectric layer comprises a silicon dioxide (SiO₂) material.

9. The method of claim 1, wherein the working electrode comprises a spongy and porous titanium nitride (TiN) working electrode with sparsely-spaced TiN columnar structures.

10. The method of claim 1, wherein the side wall comprises:
a portion surrounding a lower section of the well, and
a portion surrounding an upper section of the well, wherein the portion surrounding the upper section of the well forms an overhang above the lower section of the well, and wherein the overhang comprises the hydrophobic portion formed by a fluoropolymer material.

11. A nanopore-based sequencing device, comprising:
a conductive layer;
a working electrode disposed above the conductive layer; and
a side wall disposed above the working electrode, wherein the side wall and the working electrode form a well in which an electrolyte may be contained, and wherein at least an upper portion of the side wall comprises a hydrophobic portion formed by a fluoropolymer material.

12. The nanopore-based sequencing device of claim 11, wherein the fluoropolymer material is selected from the group consisting of Cytop and Teflon.

13. The nanopore-based sequencing device of claim 11, wherein a thickness of the hydrophobic portion formed by the fluoropolymer material is between about 10 angstroms and about 100 microns.

14. The nanopore-based sequencing device of claim 11, wherein the hydrophobic portion formed by the fluoropolymer material comprises a top horizontal hydrophobic surface above the well that facilitates the formation of a lipid bilayer that spans across the well.

15. The nanopore-based sequencing device of claim 11, wherein the hydrophobic portion formed by the fluoropolymer material comprises a vertical hydrophobic surface inside the well that facilitates the formation of a lipid bilayer that spans across the well.

16. The nanopore-based sequencing device of claim 11, wherein at least a lower portion of the side wall comprises a dielectric layer, and wherein the hydrophobic portion formed by the fluoropolymer material is disposed above the dielectric layer.

17. The nanopore-based sequencing device of claim 16, wherein a combined thickness of the hydrophobic portion formed by the fluoropolymer material and the dielectric layer is between one to ten microns.

18. The nanopore-based sequencing device of claim 16, wherein the dielectric layer comprises a silicon dioxide (SiO₂) material.

19. The nanopore-based sequencing device of claim 11, wherein the working electrode comprises a spongy and porous titanium nitride (TiN) working electrode with sparsely-spaced TiN columnar structures.

20. The nanopore-based sequencing device of claim 11, wherein the side wall comprises:
a portion surrounding a lower section of the well, and
a portion surrounding an upper section of the well, wherein the portion surrounding the upper section of the well forms an overhang above the lower section of the well, and wherein the overhang comprises the hydrophobic portion formed by a fluoropolymer material.

21. A method of constructing a nanopore-based sequencing device, comprising:
constructing a conductive layer;
constructing a working electrode disposed above the conductive layer; and
constructing a side wall disposed above the working electrode, wherein the side wall and the working electrode form a well in which an electrolyte may be contained, and wherein is at least an upper portion of the side wall comprises a hydrophobic portion formed by a fluoropolymer material.

22. The method of claim 21, wherein the fluoropolymer material is selected from the group consisting of Cytop and Teflon.

23. The method of claim 21, wherein a thickness of the hydrophobic portion formed by the fluoropolymer material is between about 10 angstroms and about 100 microns.

24. The method of claim 21, wherein the hydrophobic portion formed by the fluoropolymer material comprises a top horizontal hydrophobic surface above the well that facilitates the formation of a lipid bilayer that spans across the well.

25. The method of claim 21, wherein the hydrophobic portion formed by the fluoropolymer material comprises a vertical hydrophobic surface inside the well that facilitates the formation of a lipid bilayer that spans across the well.

26. The method of claim 21, wherein at least a lower portion of the side wall comprises a dielectric layer, and wherein the hydrophobic portion formed by the fluoropolymer material is disposed above the dielectric layer.

27. The method of claim 26, wherein a combined thickness of the hydrophobic portion formed by the fluoropolymer material and the dielectric layer is between one to ten microns.

28. The method of claim 26, wherein the dielectric layer comprises a silicon dioxide (SiO₂) material.

29. The method of claim 21, wherein the working electrode comprises a spongy and porous titanium nitride (TiN) working electrode with sparsely-spaced TiN columnar structures.

30. The method of claim 21, wherein the side wall comprises:
a portion surrounding a lower section of the well, and
a portion surrounding an upper section of the well, wherein the portion surrounding the upper section of the well forms an overhang above the lower section of the well, and wherein the overhang comprises the hydrophobic portion formed by a fluoropolymer material.
